(19) United States
(12) Patent Application Publication
(10) Pub. No.: US 2017/0172935 A1
(43) Pub. Date: Jun. 22, 2017

(54) APPLICATIONS AND TOOLS BASED ON SILICA PARTICLES COATED WITH BIOLOGICAL OR SYNTHETIC MOLECULES

(71) Applicant: NANOBIZ NANOBIYOTEKNOLOJIK SISTEMLER EGITIM BILISIM DANISMANLIK AR-GE SAN.TIC LTD. STI., Ankara (TR)

(72) Inventors: Huseyin Avni OKTEM, Ankara (TR); Veli Cengiz OZALP, Ankara (TR); Frank J. HERNANDEZ, Ankara (TR); Luiza I. HERNANDEZ, Ankara (TR)

(73) Assignee: NANOBIZ NANOBIYOTEKNOLOJIK SISTEMLER EGITIM BILISIM DANISMANLIK AR-GE SAN.TIC. LTD. STI., Ankara (TR)

(21) Appl. No.: 15/304,866

(22) PCT Filed: Apr. 18, 2014
(86) PCT No.: PCT/TR2014/000146

§ 371 Date: Oct. 18, 2016

(51) Int. Cl. A61K 9/51; G01N 33/543; G01N 33/552

(52) U.S. Cl. CPC A61K 9/5115; G01N 33/552; G01N 33/54313

(57) ABSTRACT

This invention defines gated silica nanoparticles into which chemical cargo materials or substances are embedded and of which surfaces are coated with biologically active gating molecules and apparatus employing such silica nanoparticles. The invention and the apparatus employing the invention have the potential to be used in diverse fields such as health, food and textiles. Furthermore, the applicability of the invention and the apparatus defined in this document is not limited to the above fields and can be extended to many other sectors.

### TECHNICAL FIELD

This invention defines silica nanoparticles into which chemical materials or substances are embedded and of which surfaces are coated with biologically active molecules and apparatus employing such silica nanoparticles. The invention and the apparatus employing the invention have the potential to be used in diverse fields such as health, food and textiles. Furthermore, the applicability of the invention and the apparatus defined in this document is not limited to the above fields and can be extended to many other sectors.

### THE STATE-OF-THE-ART (PRIOR ART)

Recently micro and nano sized particles have found widespread usage in many different sectors for various applications. For these ends organic (dendrimers, liposomes, polymers and virus-like particles) and inorganic (gold, semiconducting nanocrystals, magnetic particles and silica based based particles) nanoparticles are being employed.

For example, particles with magnetic properties can be used for protein and/or cell purification upon coating their surfaces with biologically active molecules (such as aptamers or antibodies)

(V. Cengiz Ozalp, Gulay Bayramoglu, Murat Kavruk, Batuhan B. Keskin, Huseyin A. Oktem, M., Yakup Arica. Pathogen detection by core-shell type aptamer-magnetic pre-concentration coupled to real-time PCR. Analytical Chemistry, 447 (2014) 119-125)

V. Cengiz Ozalp, Gülay Bayramoglu, M. Yakup Arica, H. Avni Ôktem. Design of a core-shell type immuno-magnetic separation system and multiplex PCR for rapid detection of pathogens from food samples. Appl. Microbiol. Biotechnol., in press, □ DOI 10.1007/s00253-013-5231, 2013.

Ôktem H. A., Bayramo\check{g}lu G., Özalp V. C., Arica M. Y. Single step purification of recombinant Thermus aquaticus DNA-polymerase using DNA-aptamer immobilized novel affinity magnetic beads. Biotechnology Progress, 23:146-154, 2007)

Inorganic carrier systems have certain advantages over organic carrier systems, including high mechanical stability, biocompatibility and resistance against microbial activity. Moreover, they are able to protect the cargo within from enzyme activity and factors such as pH. Due to these properties, mesoporous silica particles have gained importance in the recent years. Such particles are used for drug delivery and controlled drug release (Veli Cengiz Ozalp, Fusun Eyidogan, Hüseyin Avni Oktem, Aptamer-gated Nanoparticles for Smart Drug Delivery, Pharmaceuticals, 4(8), 1137-1157; doi:10.3390/ph408113, 2012).

In some applications, drug loaded particles are targeted specifically to diseased tissue and/or cells. Upon reaching target tissue/cells, particles release the drug molecules stimulated by factors such as temperature, radiation, magnetic fields, and pH change and therefore drug molecules interact specifically with the target area.

For targeting, particle surfaces are coated with bioreceptor molecules (such as antibodies or aptamers) specific to the target tissue or cells. Therefore, drug loaded particles gather only where the drug therapy is required and side effects to healthy cells and tissues are prevented. Such strategies are especially important in cancer therapy.

In addition to drug delivery, these particles can be tagged (for instance with fluorescent molecules) and can be used for the detection, diagnosis and monitoring of the targeted tissue and cells.

Nowadays, in addition to targeted drug delivery, new smart systems, which release their cargo (drug, signalling molecule, etc.) when a targeted biological agent is present, are required.

In the literature there are only a few examples of such studies. In a 2010 paper, it was shown that antimicrobial chemicals and fluorescent molecules were selectively secreted by S. aureus but that E. coli cells did not secrete the particle and were not affected by the chemical. (Jin Zhou, Andrew L. Loftus, Geraldine Mulley, and A. Toby A. Jenkins. J. AM. CHEM. SOC. VOL. 132, NO. 18, 6566-6570, 2010). Lipid vesicles used in this study provide a limited field of application because lipid vesicles have low selectivity and therefore are limited in terms of use.

There are very important applications where these systems can be employed. For instance, by using smart systems which release the antibiotic or antiviral substances they carry, when target bacteria and/or virus are present, it will be possible to prevent the emergence of multidrug resistant microorganisms (super bugs). Similarly, smart systems which release the anticancer drugs upon encounter with cancer cells can maximize the damage on the cancer cells and minimize hazardous effects on healthy cells.

Furthermore, it is possible to utilize these smart systems in molecular diagnosis. With our invention it will be possible to produce systems which will give a signal only when a particular target microorganism is present within a test sample.

As of now, carrier systems with a selectivity of a level described above has not been achieved. Our invention brings an important novelty to this field and defines a system which will release its cargo only when a target analyte or cell is present. Additionally, some fields and apparatus in which this system can be used are defined.

### BRIEF DESCRIPTION OF THE INVENTION

With this invention, various molecules are loaded within silica nanoparticles and the surfaces and nanopores of nanoparticles are coated with biological molecules to prevent the leaking of the cargo molecules. Molecules used in surface coating and gating include, modified or unmodified nucleic acids (single or double chain DNA or RNA molecules), lipids, peptides, proteins, carbohydrates or synthetic molecules. These gating molecules interact with a specific molecules (such as enzyme, single chain nucleic acids or other metabolites) secreted by the target cell or tissue. As a result of this interaction gating molecules undergo degradation or a structural change or removed from the nanoparticle surface and lose their ability to gate. Consequently, the carrier releases its cargo. As a result, depending on the type of cargo, therapy, diagnosis or simultaneously therapy and diagnosis can be achieved.

### DEFINITION OF THE FIGURES EXPLAINING THE INVENTION

Figures demonstrating the principle of the invention, usage of silica nanoparticles coated with biologically active molecules, and the explanation of images are given below.

FIG. 1. Biomolecule coated silica nanoparticles releasing their cargo, only when the target cell is present.

FIG. 2. Use of silica nanoparticles in detection of a target molecule.

FIG. 3. The ability of the particles to detect a target cell, when bound to a solid substrate (for example a lateral flow test strip).

FIG. 4. Nanoparticles, of which surfaces are coated with nucleic acids, detecting a target nucleic acid sequence.

FIG. 5. Selective destruction of a target cell by silica nanoparticles embedded in a fiber or gel-like matrix.

FIG. 6. Detection and selective destruction of a target cell by silica nanoparticles embedded in a fiber or gel-like matrix.

FIG. 7. Application of the matrix into which the particles are embedded to a first aid plaster.

FIG. 8. Detection and selective destruction of target organisms by silica nanoparticles embedded in a textile.

FIG. 9. The ability of the particles to detect multiple types of target cell simultaneously, when bound to a solid substrate (for example a lateral flow test strip).

FIG. 10. Detection of the target cell in a liquid medium by the selectively coated particles only when a specific metabolite is secreted by the target cell.

FIG. 11. Detection of target cells in a liquid sample by the selectively coated particles.

FIG. 12. Demonstration of selectively coated nanoparticles releasing the signal molecule (fluorescent molecule) they carry only when an enzyme (MN) secreted by the target cell is present PBS solution or blood serum does not cause any release.

FIG. 13. Selectively coated particles demonstrate the ability to get activated at a lower antibiotic concentration and kill target microorganisms (WT) when they are present. When non-target (mutant) microorganisms are present, the activity can be observed at a higher antibiotic concentration. In the graph, lower absorbance values indicate killing of microorganisms.

### ELEMENT NUMBERS

1. Silica nanoparticle
2. Cargo
3. Gating molecules
4. Hole
5. Target
6. Target cell
7. Non-target cell
8. Signalling molecule
9. Nucleic acid
10. Nucleic acid with the complementary strand
11. Molecule, which produces a signal upon interaction with the signalling molecule
12. Signal visible with the unaided eye
13. A molecule with the ability to degrade the gating molecules (14) on the control particles
14. Gating molecule for the silica particles in the control site
15. Sample loading site
16. Site on the strips which contain the molecule, which is able to degrade the gating molecules on the particles
17. Analysis (test) site
18. Control site
19. Sample absorption site
20. Molecule, which activates the gating molecules on the control particles
21. Gating molecule for the control particles
22. Textile or gel-like structures
23. Wound
24. Matrix with fabric structure
25. First aid plaster
26. Matrix of first aid plaster
27. Transparent layer

### DEFINITION OF THE ELEMENTS AND PARTS COMPOSING THE INVENTION

In order to clearly define the components and elements of the applications developed for this invention, parts in the figures are separately numbered and the definitions of each numbered part is given below.

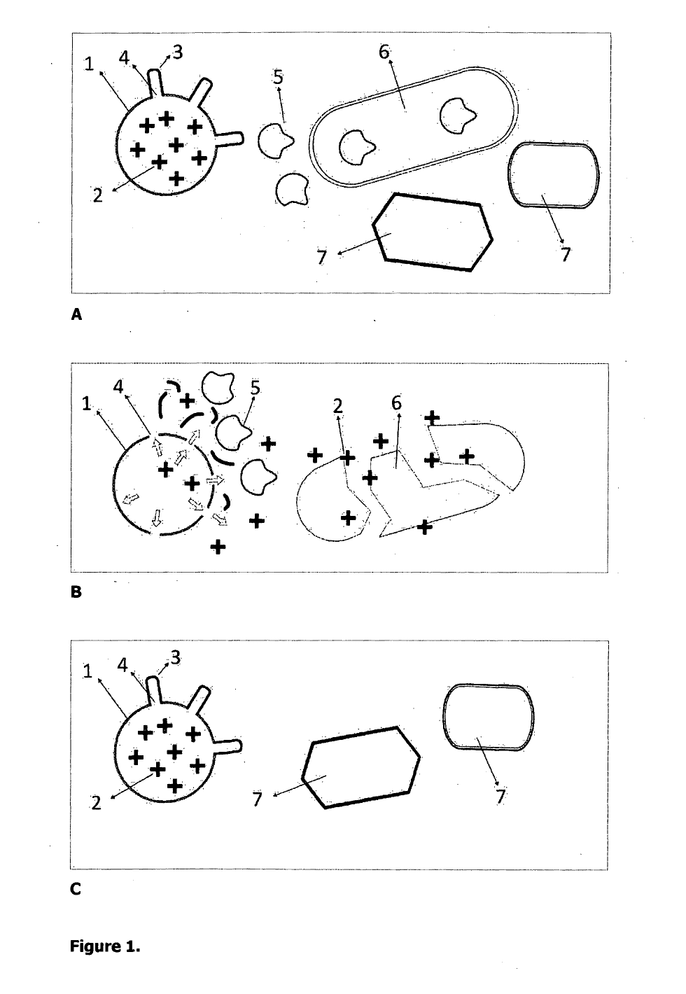

This invention is based on silica nanoparticles, which have a potentially broad spectra of biological applications. FIG. 1 explains the principle behind which the silica nanoparticles release their cargo when the target cell or tissue is present. In this application, silica particles (1) loaded with various types of cargo (2) and coated with gating molecules (3) release their cargo only when the molecules (5) released by the target cells (6), degrade the gating molecules (3) on the silica nanoparticles (1) (FIG. 1-B). This provides the release of the cargo molecules to the medium. If the cargo is an antibiotic or an antimicrobial substance the target cell (for instance a bacterium) can be destroyed. (FIG. 1-B). If a cell (7) does not posses the ability to secrete molecules which can degrade the gating molecules, particles do not release their cargo (FIG. 1-C). This feature of our invention provides the release of the antibiotic cargo only when the target bacteria are present and therefore prevent multidrug resistance due to excessive antibiotic usage. Furthermore, non-target, beneficial microorganisms will be unaffected by the antibiotics. In another yet similar application, particles loaded with anticancer drugs and coated with gating molecules which are only degraded and/or removed by molecules secreted by cancer cells, will be effective against only cancer cells and prevent side effects to healthy cells.

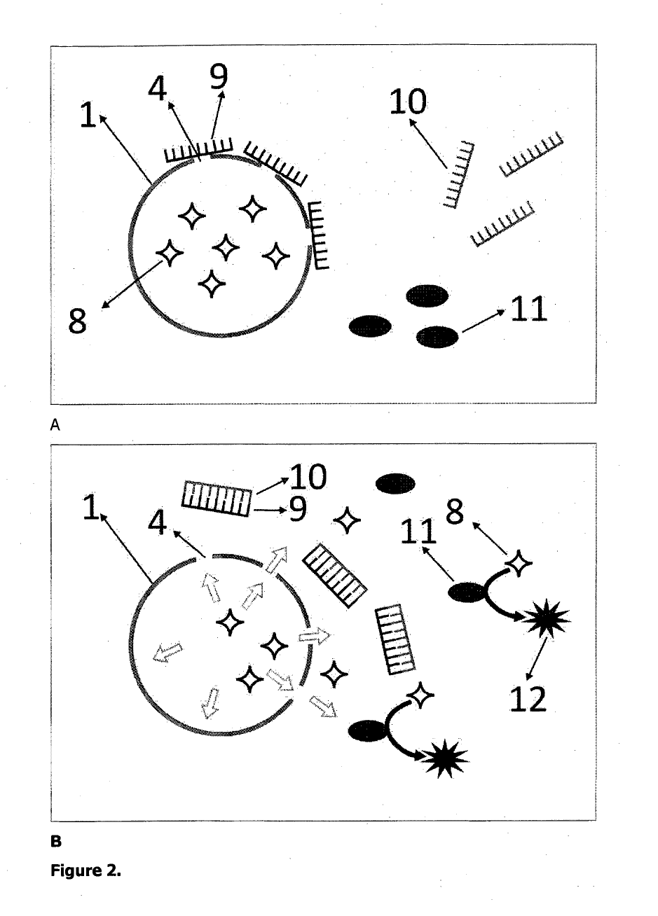

Another important platform for our invention is in diagnosis platforms. In this kind of applications, particle (1) surfaces are coated with a defined sequence of nucleic acid molecules (9) for gating. Particles are loaded with a signalling molecule (8). Outside the particles, another molecule which interacts with the signalling molecule to produce a detectable signal, is present (11) (FIG. 2-A). When a nucleic acid (DNA or RNA) (10), which has a sequence complementary to the sequence of the nucleic acid molecules gating the particles (9) is added to the medium, the gating molecule (9) and the added molecule (10) hybridize with each other, which in turn makes the gating molecule lose its gating ability (4) and therefore cause the release of the signalling molecule (8). The signalling molecule (8) then interacts with the signal developing molecule (11) (for example TMB as the signalling molecule (8) and Horse Radish Peroxidase (HRP) enzyme as the signal developing molecule (11)), which results in a detectable signal (FIG. 2-B). Depending on the nature of the signalling molecule, it is possible to produce a visible or fluorescent signal without using a signal developing molecule (11).

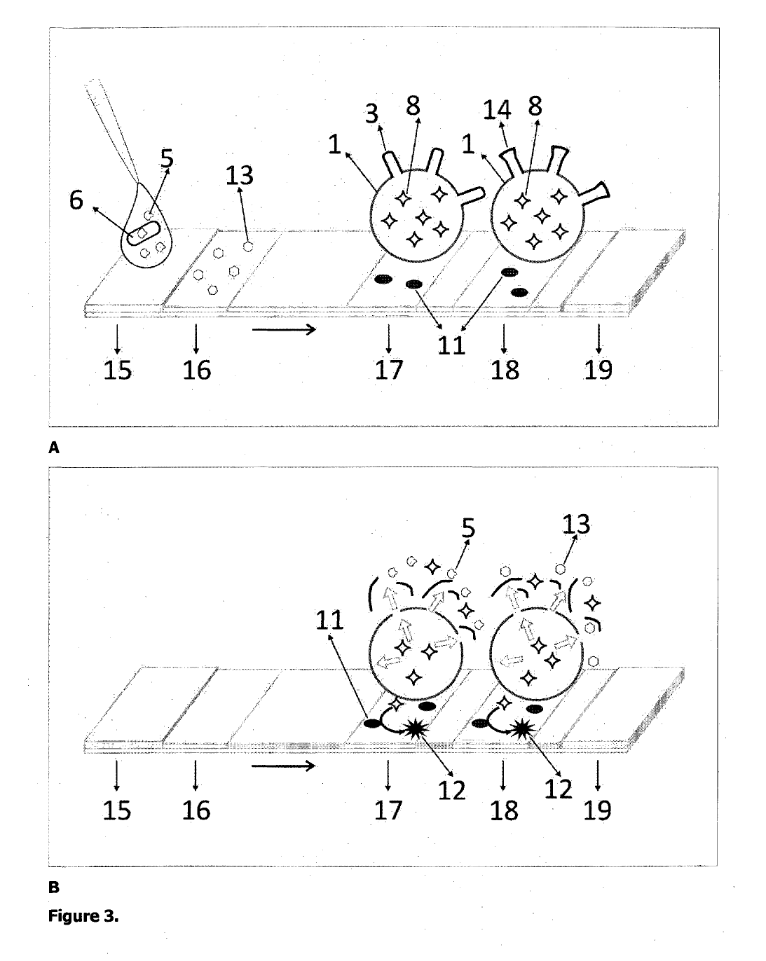

These particles can also be applied to immunochromatographic lateral flow test strips, where target cells or molecules can be detected. (FIG. 3).

For example, a liquid sample containing the target cell (6) and a molecule specific to the target cell (4) is dropped on the sample loading site (15) (FIG. 3-A). As the sample proceeds towards the analysis (test) site (17), it passes through a site (16), which contains a molecule (13) with the ability to degrade or displace the gating molecules (14) on the particles present at the control site (18). When the sample reaches analysis (test) site (17), the gating molecules (3) on the particles at this site are degraded by the molecules (5) secreted by the target cell (6) in the sample. This causes the release of the signalling molecule (8), which in turn interacts with the signal developing molecule (11) on the analysis (test) site and produces a signal. Depending on the type/nature of the signalling molecule, it is possible to produce a visible or fluorescent signal without using a signal developing molecule (11). As a result, a signal develops on the analysis (test) site, if the target cell is in the sample applied to the lateral flow assay strip. The sample applied to the strip passes through the control site (18) before it finally reaches the sample absorption site (19). Control site, is designed in a way that, there is a signal to control that the strips are working properly, whether or not the target cells and/or molecules are present in the sample. When the sample reaches the control site (18), the molecule present in site 16 is carried along. By this, gating molecules (14) coated on the surfaces of the particles in the control site (14) are degraded or displaced by the molecule (13), which specifically degrades the gating molecules. This releases the signalling molecule (8) from the particles, which in turn interacts with the signal developing molecule (11) and results in signal production. Depending on the type/nature of the signalling molecule, it is possible to produce a visible or fluorescent signal without using a signal developing molecule (11) (FIG. 3-B). In the case of a visible signal, the results can be visualised by unaided eye, without the necessity to use an optical device.

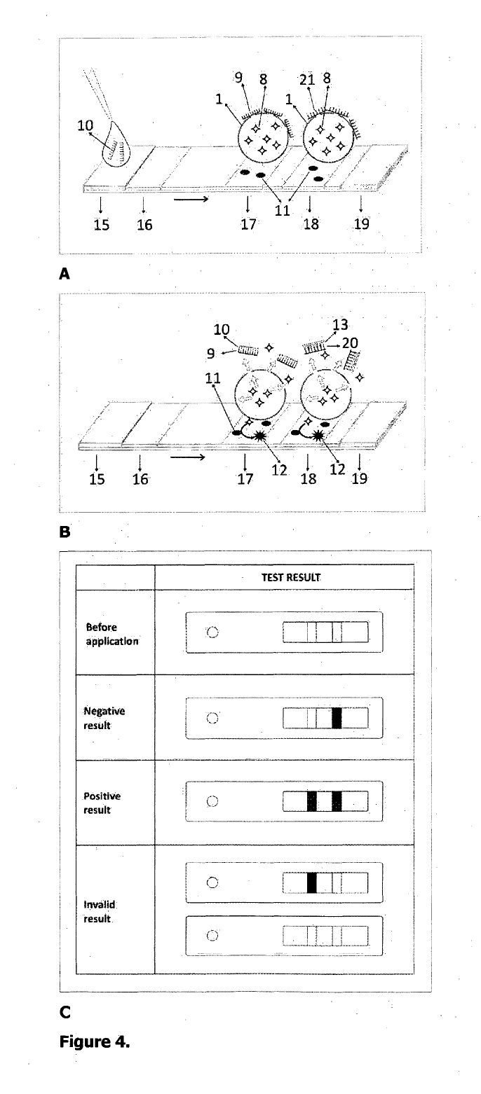

A similar apparatus can be used for the detection of a target nucleic acid molecule (FIG. 3-B). Sample containing the target molecule (10) is dropped to the sample loading site (15). Sample moves to the analysis (test) site by capillary movement. In the analysis (test) site there are particles coated with gating nucleic acid molecules (9) which are complementary to the target molecule (10)'s nucleic acid sequence. This results in the hybridization of the target molecule (10) and the gating molecule (9) and release of the hybrids from the particle surface. This results in the opening of the pores on the particle surface. Signalling molecule (8) are released from the particles, and interact with the signal developing molecule (11) on the analysis (test) site (17), resulting in the formation of a detectable signal. Depending on the type/nature of the signalling molecule, it is possible to produce a visible or fluorescent signal without using a signal developing molecule (11) (FIG. 4-B). As a result, a signal develops on the analysis (test) site, if the target nucleic acid is in the sample applied to the lateral flow assay strip.

The sample applied to the strip passes through the control site (18) before it finally reaches the sample absorption site (19). Control site, is designed in a way that, there is a signal to control that the strips are working properly, whether or not the target nucleic acids are present in the sample. When the sample reaches the control site (18), the molecule (20) present in site 16 is carried along. By this, the gating molecules (21) coated to the surfaces of the particles on the control site (18) hybridize with the molecule (20) complementary to the gating molecules and leave the surface of the particles opening the pores (4). This releases the signalling molecule (8) from the particles, which in turn interacts with the signal developing molecule (11) and results in signal production. Depending on the type/nature of the signalling molecule, it is possible to produce a visible or fluorescent signal without using a signal developing molecule (11) (FIG. 4-B).

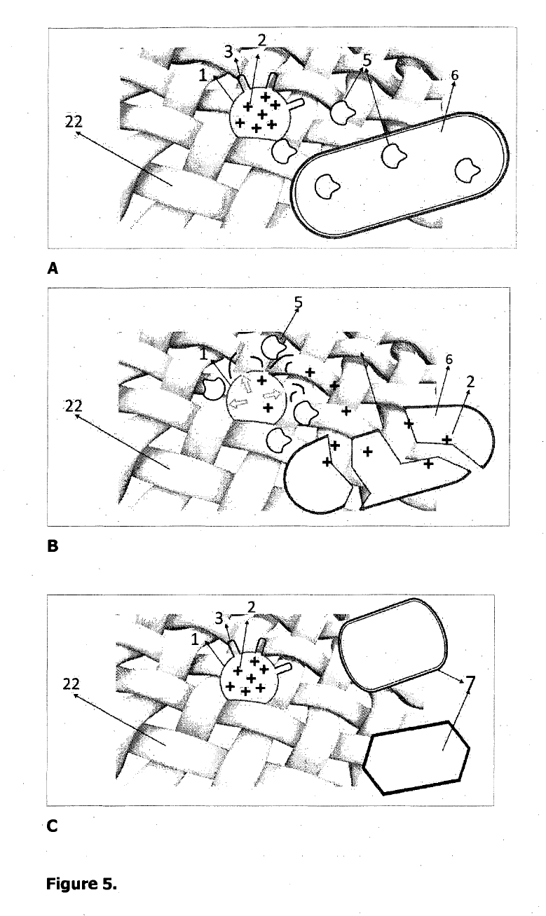

By this apparatus, detection of single point or multiple point mutations on the target DNA molecules is also possible. In this type of analysis, target sequences with mutations are analysed with particles coated with complementary wild type sequences (9) and according to the signal development on the test site, the presence of mutations is determined (FIG. 4-C). In another application, coated particles are embedded into textile or gel-like matrices (22) (FIG. 5-A). In this application, when the particle embedded matrices come in contact with the target cells (6), the molecule (5) secreted by the cells, which has the property to degrade the gating molecules (3) coated on the particles, degrades the gating molecules to release the cargo (2) of the particles. If the cargo is an antimicrobial substance (antibiotics, antifungal agents, or chemicals like sodium azide), it is possible to eliminate the target organism (FIG. 5-B). In the presence of the non-target organism (7), cargo is not released (FIG. 5-C).

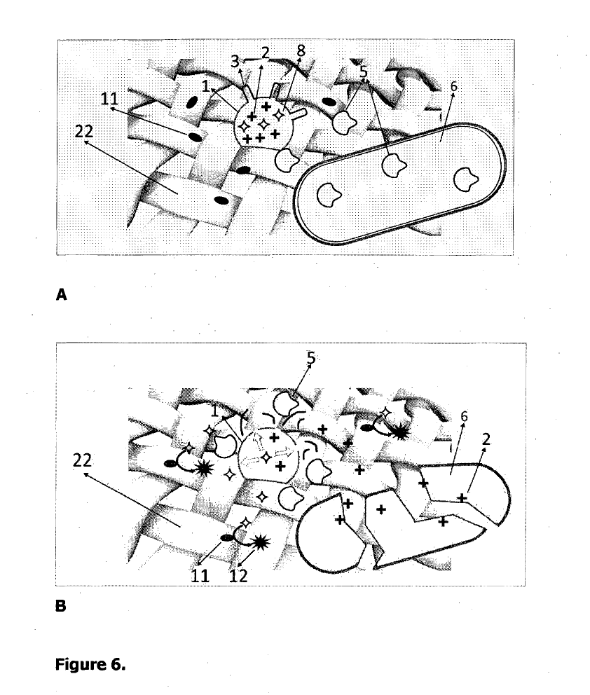

In this kind of application, depending on the functionality of the cargo (2 and 8) loaded into the particles, different outcome schemes can be obtained. For example, by loading the particles with an antimicrobial molecule and a signalling molecule, it is possible to kill the target cells and visualize the presence of the target cell at the application site (FIG. 6). When the target cell (6) is present, a molecule (5) secreted by the target cell specifically degrades the gating molecules (3) coated onto the particles embedded in the matrix (22). This opens the pores (4) on the particles, which in turn causes the release of the cargo (2 and 8). In the case that one of the molecules in the cargo is antimicrobial, the target cell is eliminated (FIG. 6-B). Simultaneously, the signalling molecule (8) interacts with the signal developing molecule (11) to produce a signal (FIG. 6-B). Depending on the type and nature of the signalling molecule (for instance a dye molecule visible by the unaided eye), visible signal can be produced without a signal developing molecule (11). In this application, the target cell can be a bacterium, a fungus, a yeast or a cancer cell.

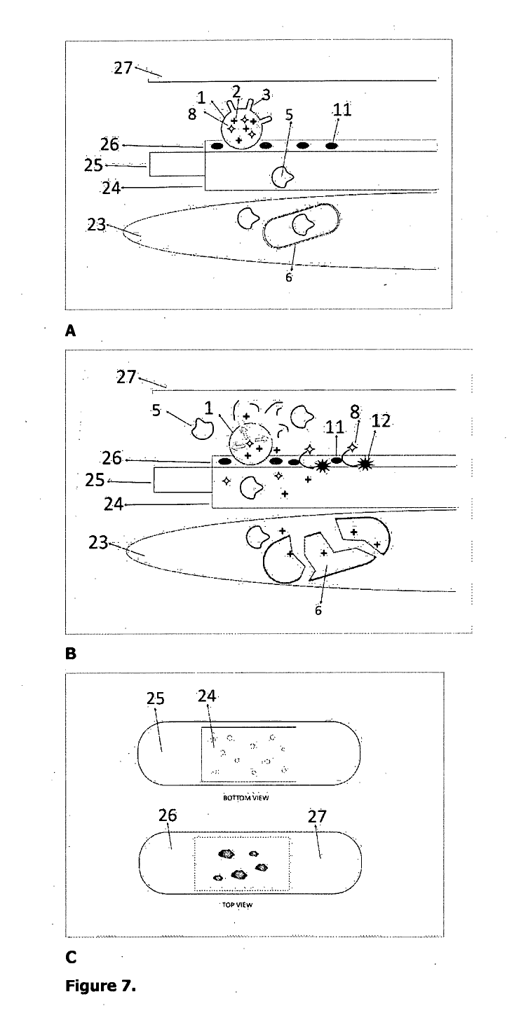

The invention explained above can be used in first aid plasters (FIG. 7). A fabric matrix (24) as shown in FIG. 6 can be combined with first aid plasters (25). The outer layer of the plaster is covered by a transparent layer (27) in order to visualize the signal. In the case of an infection, a visual signal is produced (12), which can be detected by unaided eye. If an infection takes place in the wounded area (23), a molecule (5) secreted by the target organism (6) degrades the gating molecule (3) coated on the particles (1) embedded into the matrix (26) and both the antimicrobial cargo (2) and the signalling cargo (8) are released. This eliminates the target microorganism in the wounded area and the signal molecule either on its own or by interacting with a signal developing molecule (11) form a visible signal (12) (FIG. 7-B). Therefore, it is possible to both heal and detect an infection in an area where such first aid plasters are applied.

Our invention can be extended to other textile products: By embedding such particles into baby diapers, it will be possible to detect urinary tract infections in babies very fast and selectively. Another possibility is to embed such particles to socks, by which it will be possible to detect fungal infections of feet by a visible signal and eliminate fungi using antifungal cargo. It is also possible to use our invention in hygiene pads and/or underwear to detect and heal genital tract infections. By using our invention in plasters for burns, infections in burns can be detected and healed very rapidly, for an effective treatment without any delay. Alternatively the particles can be embaded in tissue papers to identify contaminated surfaces and infectious in body fluids such as salavia and mucus.

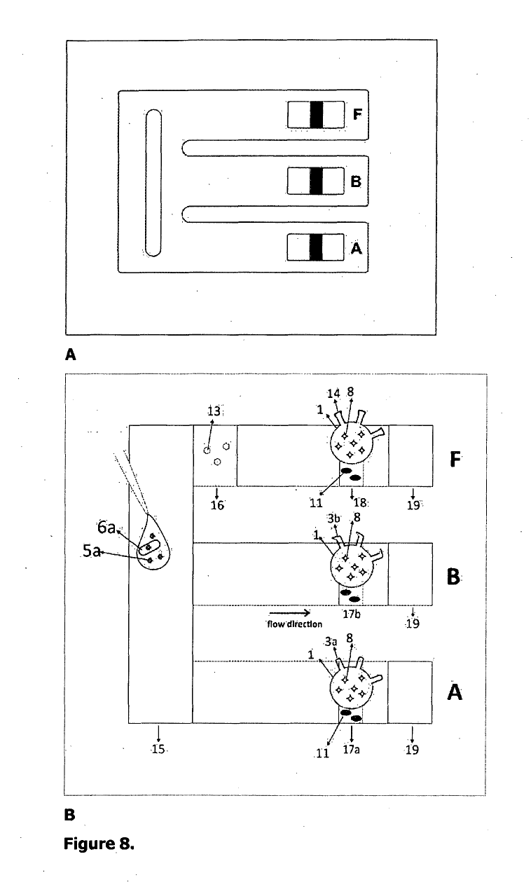

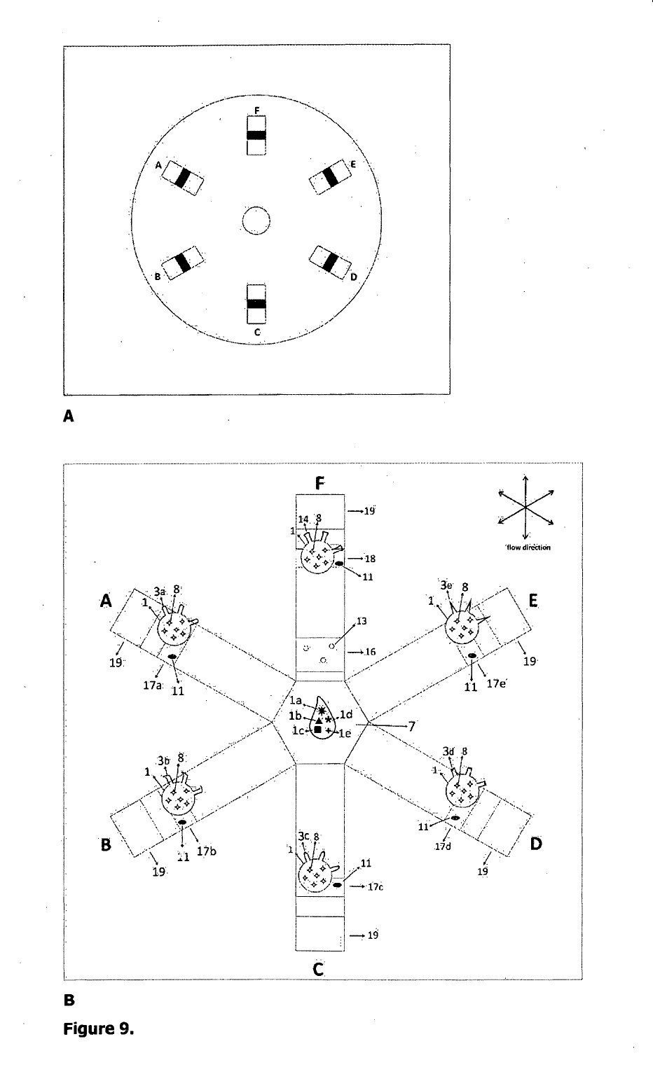

Our invention can be utilized in lateral flow assay strips for the simultaneous detection of more than one type of cells. Apparatus for the detection of 2 or five different types of target organisms and their working principle are given in FIGS. 8 and 9, respectively. By increasing the number of channels in the test cards, the number of target organisms to be detected can be increased. F channel is the control channel for checking whether the test card works properly. For the validity of the test a signal is expected in the F channel, whether or not a target organism is present. For this end, a molecule (13), which can degrade the gating molecules (14) on the silica particles on site 18, are placed on the site 16. Sample to be tested is applied to sample loading site (19). As the sample progresses towards the sample absorption site (19), molecule 13 is carried along towards site 18. When molecule 13 reaches site 18, it degrades or displaces the gating molecules (14) coated onto particles and cause the release of the signalling molecule. As explained before this release results in the formation of detectable signal. This verifies the proper functioning of the test platform. If target organisms (6a and 6b) are present in the sample, in A and B channels signals are observed. When the sample reaches the analysis (test) site (18) at A and B channels, specific molecules (5a and 5b) secreted by the target organisms degrade the gating molecules (3a and 3b) coated on the nanoparticles and cause the release of the signalling molecules (8). Signalling molecules either interact with the signal developing molecules (11) in the analysis (test) site to develop a signal or signalling molecules can have intrinsic fluorescent properties to directly produce signal. Either way, if the target organisms are present in the sample, a signal (visible or fluorescent) is detected at the channels specific to each organism

FIG. 9-A describes an apparatus capable of detecting 5 different types of target organisms simultaneously. A sixth channel (F channel) is present for the verification of the test. This F channel works with the same principle as described in FIG. 8. There are five test channels (A-E) for the detection of target organisms. FIG. 9-B shows the working principle of this apparatus. Sample is applied to the sample loading site (7) in the middle of the apparatus. In the test channels, silica particles coated with gating molecules (3a-3e), which are specifically degraded by the molecules (1a-1e), secreted by the target organisms are immobilized. When the secreted molecules (1a-1e) reach the analysis (test) areas, gating molecules (3a-3e) are degraded or displaced which releases the signalling molecules (8). Signalling molecules either interact with the signal developing molecules (11) in the analysis (test) site to develop a signal or signalling molecules can have intrinsic fluorescent properties to directly produce signal. By increasing the number of the channels on the apparatus, the number of different test organisms to be detected can be increased as well.

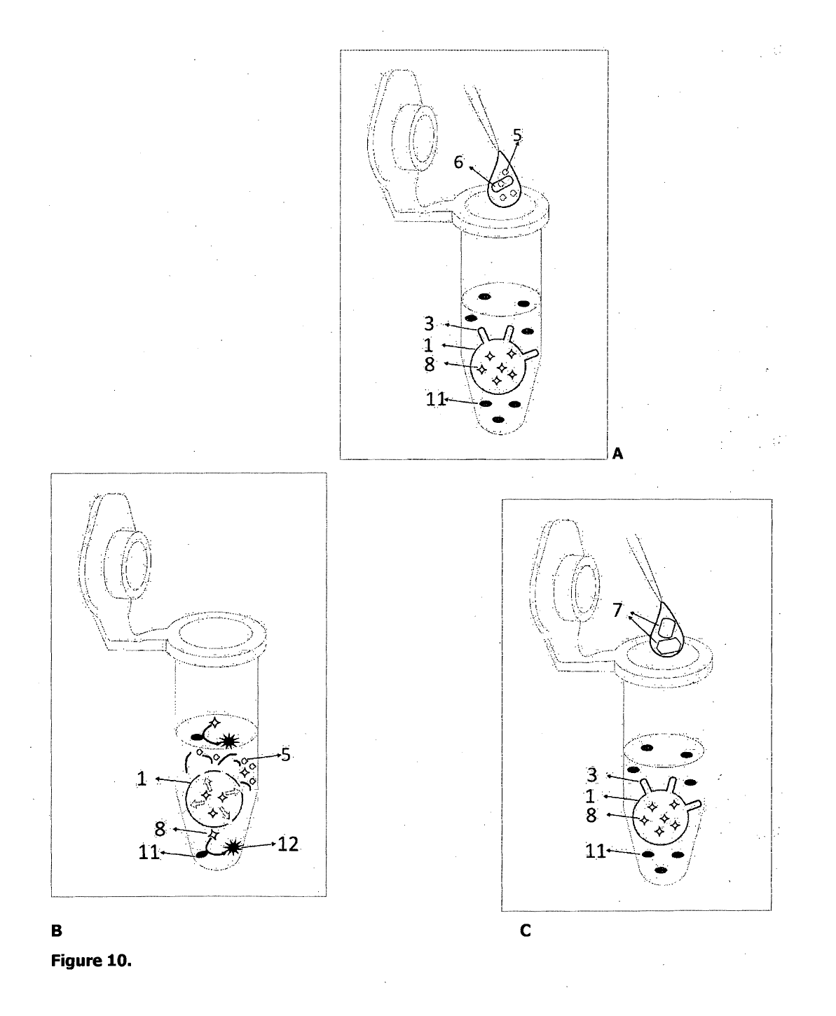

In addition to lateral flow test assays, our invention can function in liquid media for the detection of target organisms (FIG. 10). Target organism (6) and the molecule (5) secreted by the organism is added to the liquid test medium containing the silica particles (1). Silica particles are coated with gating molecules (3), which are degraded by the molecule (5) secreted by the target organism and are loaded with signalling molecules (8). When the sample added to the test environment contains the target organism (6) and hence the molecule (5) secreted by the target organism, gating molecules (3) coated onto silica nanoparticles are degraded and the signalling molecule (8) is released to the medium, which in turn interacts with the signal developing molecule (11) to develop a signal (FIG. 10-B). Alternatively, by choosing a fluorescent signalling molecule (8), a direct signal can be obtained. If the target organism is absent in the sample added to the test medium, no signal is observed (FIG. 10-C).

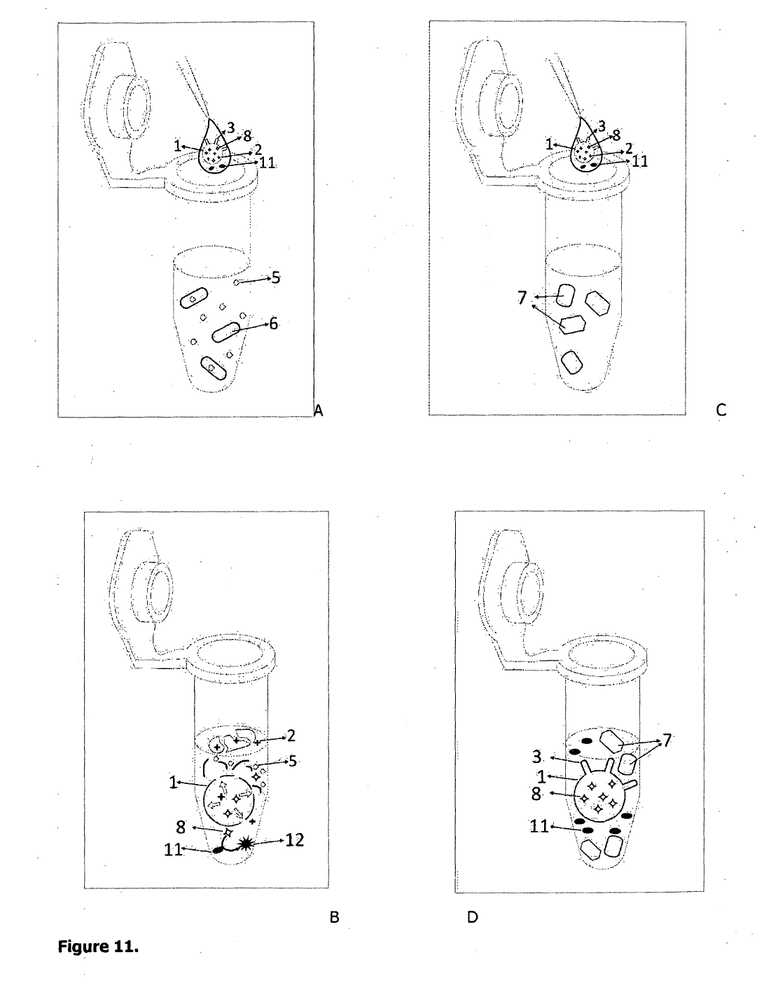

In another variant, target microorganisms present in a liquid medium can be detected fast and simply (FIG. 11). In this variant, to a liquid medium containing the sample, surface modified silica nanoparticles are added (FIG. 11-A). The added silica particles are loaded with signalling molecules (8) and are coated with gating molecules (3) which are specifically degraded or displaced by the molecules (5) secreted by the target organism (6). Silica particles are added together with a signal developing molecule (11). If the target organisms are present in the sample, the molecules (5) secreted by the target organism (6) degrade or displace the gating molecules (3) coated on the silica particles and the signalling molecule (8) is released. Upon the interaction of the signalling molecule (8) with the signal developing molecule (11) a detectable signal is produced (FIG. 11-B). Alternatively, by choosing a fluorescent signalling molecule (8), a direct signal can be obtained. If the target organism is absent from the sample added to the test medium, no signal is observed (FIG. 10-C). In this system; by adding particles prepared specifically for different type of organisms, the spectrum with which the target organisms are monitored can be broadened. Furthermore, by loading antimicrobial agents or drugs into the particles, detection and elimination of microorganisms can be carried out simultaneously.

Our invention is capable of detecting target organisms in any type of liquid media quite fast. For example with our invention it is possible to detect microorganisms in saliva and sputum very fast and without the need of using any instrument. For example fast and practical tuberculosis diagnosis will be possible with our invention. Detection and identification of the bacteria playing a role in upper respiratory tract infections is another opportunity which can be realized using our invention.

The technology developed is applicable for the drug delivering targeted particles. Such drug delivering particle surfaces can be modified with molecules that are selectively degraded or displaced upon encounter with the target cells and with specific markers such as aptamers or antibodies for targeting, so that the particles loaded with drug molecules are directed to the target cells, and they release their cargo if and only if they encounter the target cells. This would minimize side effects to healthy tissues or to reduce the chance of developing multidrug resistant cells.

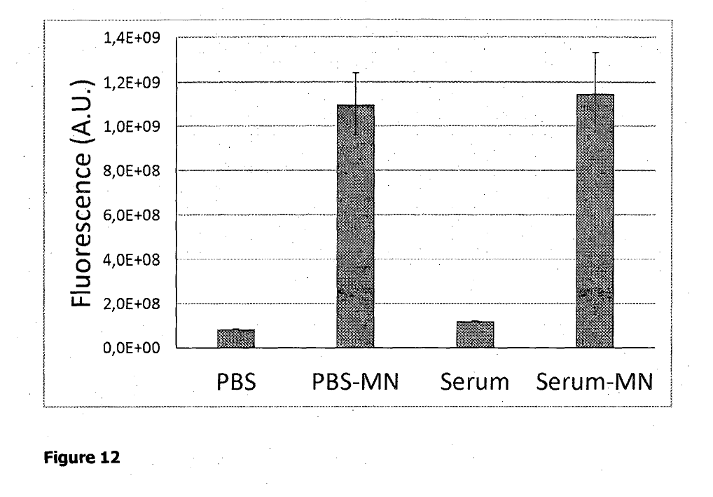

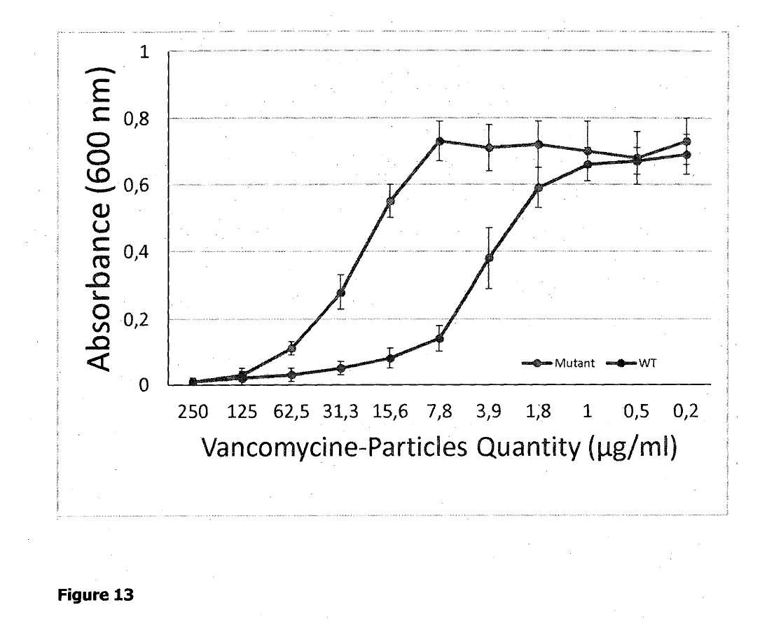

Experimental results relating to our invention are given in FIGS. 12 and 13. The explanation of the results is as follows.

In FIG. 12, results of an experiment carried out using the enzyme micrococcal nuclease (MN), secreted specifically by the bacterium Staphylococcus aureus are illustrated. In this experiment silica nanoparticles were loaded with a fluorescent chemical (Rhodamine B) and were coated with a nucleic acid molecule bearing a sequence (mCmUmCmGTTmCmGmUmUmC-m indicates 2′O-Methylated nucleotides) specifically degraded by the MN enzyme. The principle was based on the increase in the fluorescent signal due to Rhodamine B release from pores of the silica nanoparticles upon degradation of the gating nucleic acid molecule by the MN enzyme. When, the same nanoparticles were treated with Phosphate Buffered Saline (PBS) or blood serum devoid of MN enzyme; as expected, there was no increase in the fluorescence signal. These findings are of paramount importance for showing that the nanoparticles are stabile in blood serum and do not release their cargo in the absence of the target organism. Therefore, it is concluded that, nanoparticles loaded with different drugs (for example, antibiotics, antimicrobial agents, antifungal agents, chemotherapy drugs against cancer, etc.) and coated with different gating molecules specific for different cells can circulate in the body without releasing their cargo and that they release their cargo only when they encounter their respective target cells. For instance, a nanoparticle loaded with antibiotics will release its cargo only when an infection (i.e. the target microorganism) is present. Therefore, the mutually beneficial microorganisms in the gastrointestinal tract will not be unnecessarily affected by the antibiotics. Similarly, particles loaded with anticancer drugs will release their cargo only when they encounter cancer cells to kill cancer cells and healthy cells will, therefore, be minimally affected by such drugs. This brings about three important outcomes: Multidrug resistance of bacteria thus formation of super bugs can be prevented, Damage to normal flora of the human body will be reduced, side effects of anticancer drugs to healthy cells will be minimized.

In another experimental setup, similar nanoparticles were loaded with an antibiotic (vancomycin) and were coated with a nucleic acid molecule of which sequence is specifically cleaved by the MN enzyme. These particles were used to determine minimum inhibitory concentration (MIC) in cultures of S. aureus secreting MN and S. aureus mutant not secreting MN. As expected, the culture of the S. aureus secreting MN demonstrated a much lower MIC value (FIG. 13). According to these experimental findings, our invention can be utilised in the specific detection and elimination of target cells.

In another experimental design, silica nanoparticles were loaded with TMB (3,3,5,5′-tetramethylbenzidine) molecules and coated with single chain DNA molecules, which were 20 nucleotides long. These particles were immobilized onto test lines of lateral flow test strips as illustrated in FIG. 4. Horse radish peroxidase (HRP) enzyme, which interacts with TMB and produces a blue colour, was placed nearby the test line. When single stranded DNA, complementary to the single chain DNA coated onto silica nanoparticles, were loaded to the test strip, a blue coloration in the test line was observed. This proves that the single chain DNA molecules hybridize with the single chain DNA molecules coated onto silica nanoparticles and that the hybridization causes the opening of the pores on the silica nanoparticles. Subsequently TMB is released from the nanoparticles and interacts with HRP to produce a blue colour. As control, the same test strips were run with only buffer (devoid of nucleic acids) or with DNA molecules not complementary to the DNA molecules coated onto the nanoparticles. In both cases no colour formation was observed. Therefore, we conclude that with our invention, it is possible to detect specific DNA sequences in a given aqueous sample. In this context our invention will prove useful in the detection of single nucleotide polymorphisms (SNP) or multiple mutations. Additionally, our invention can be used in the detection of DNA molecules amplified by polymerase chain reaction (PCR). In this type of application, nanoparticles are coated with single stranded DNA sequences complementary (fully or partially) to the DNA sequence of DNA molecules amplified by PCR and loaded with a signalling molecule. The double stranded DNA molecules produced as a result of PCR are denatured to single strands using an appropriate method (elevated temperatures or chemicals such as NaOH). Afterwards, silica nanoparticles are added. If the complementary sequences are present among PCR products, due to hybridization the signalling molecule within the nanoparticles are released and a signal is observed.

Our invention is not limited to the applications in this document and can be used for various other applications in fields like medicine, veterinary sciences, food, environment, agriculture, water analyses, defence industry, border security and homeland security.

1. A silica nanoparticle characterised by comprising gating molecules for blocking the pores on its surface and pores that are opened as the gating molecules are degraded or displaced when a target is encountered and the gating molecules interact with the target.

2. A silica nanoparticle according to claim 1, characterised in that it carries a cargo

3. A silica nanoparticle according to claim 2, characterised in that the cargo has therapeutic properties.

4. A silica nanoparticle according to claim 3, characterised in that the cargo is antibiotic or antiviral.

5. A silica nanoparticle according to claim 2, characterised in that the cargo has diagnostic properties.

6. A silica nanoparticle according to claim 5, characterised in that the cargo is at least one signal producing molecule.

7. A silica nanoparticle according to claim 6, characterised in that the cargo interacts with another molecule and produces a signal.

8. A silica nanoparticle according to claim 6, characterised in that the cargo produces a visible or fluorescent signal on its own.

9. A silica nanoparticle according to claim 2, characterised in that the cargo is a combination of at least one therapeutic molecule and one diagnostic molecule.

10. A silica nanoparticle according to claim 1, characterised in that the gating molecules covering the pores on its surface are selected from a group comprising modified nucleic acids, unmodified nucleic acids, lipids, peptides, proteins, carbohydrates and synthetic molecules.

11. A silica nanoparticle according to claim 1, characterised in that the target is a cell, enzyme, single chain nucleic acid or metabolite.

12. A silica nanoparticle according to claim 1, characterised in that the gating molecules is a nucleic acid and the cargo is a signal producing molecule.

13. A silica nanoparticle according to claim 12, characterised in that it is used in the detection of nucleic acids with a sequence complementary to the gating molecule.

14. A silica nanoparticle according to claim 12, characterised in that it is used in the detection of nucleic acids with a sequence not complementary (mutated) to the gating molecule.

15. A silica nanoparticle according to claim 2, characterised in that it is embedded in a matrix.

16. A silica nanoparticle according to claim 15, characterised in that the matrix is fiber, membrane or gel.

17. A silica nanoparticle according to claim 15, characterised in that the matrix is first aid plaster.

18. A silica nanoparticle according to claim 15, characterised in that the matrix is burn plaster.

19. A silica nanoparticle according to claim 15, characterised in that the matrix is sanitary pad.

20. A silica nanoparticle according to claim 15, characterised in that the matrix is textile.

21. A silica nanoparticle according to claim 20, characterised in that the textile is a sock.

22. A silica nanoparticle according to claim 20, characterised in that the textile is underwear.

23. A silica nanoparticle according to claim 15, characterised in that the matrix is a diaper.

24. A silica nanoparticle according to claim 15, characterised in that the matrix is paper

25. A silica nanoparticle according to claim 15, characterised in that the matrix is a lateral flow assay test strip.

26. A silica nanoparticle according to claim 1, characterised in that it is used in liquid sample.
